(19) United States
(12) Patent Application Publication
(10) Pub. No.: US 2010/0008987 A1
(43) Pub. Date: Jan. 14, 2010

(54) Modified Release Pharmaceutical Composition of Bupropion Hydrochloride

(76) Inventors: Pasula Basavaiah Chowdary, Andhra Pradesh (IN); Anuj Kumar Fanda, Himachal Pradesh (IN); Gour Mukherji, Haryana (IN); Maulik Kiritkumar Panchal, Gujarat (IN); Piyush Kumar Kansagra, Gujarat (IN)

Correspondence Address: COHEN, PONTANI, LIEBERMAN & PAVANE LLP, 551 FIFTH AVENUE, SUITE 1210, NEW YORK, NY, 10176, US

(21) Appl. No.: 12/438,281

(22) PCT Filed: Aug. 21, 2007
(86) PCT No.: PCT/IN2007/000357

§ 371 Date: Feb. 20, 2009

(30) Foreign Application Priority Data
Aug. 21, 2006 (IN) 1877/DEL/2006

(51) Int. Cl. A61K 9/24; A61K 31/138

(52) U.S. Cl. 424/472; 427/2.21; 514/646

(57) ABSTRACT

A delayed extended release pharmaceutical composition includes a compressed core containing an effective amount of bupropion or its pharmaceutically acceptable salt, a water-attractant polymer. The core is preferably devoid of a stabilizer. The core is surrounded by an extended release layer, which is free of plasticizer and pore-forming agent. The extended release layer is surrounded by a delayed release layer. Alternating coats of extended release layer and delayed release layer may follow. A preferred extended release layer includes ethylcellulose and hydroxypropyl cellulose or hydroxypropyl methylcellulose and a preferred delayed release layer includes methacrylic acid copolymer and hydroxypropyl methylcellulose phthalate, lactose and a combination of triethyl citrate and polyethylene glycol and talc. A method of preparing the delayed extended release bupropion hydrochloride containing pharmaceutical composition is also disclosed.

### PRIORITY CLAIM

This is a U.S. national stage of PCT Application No. PCT/IN2007/000357, filed on Aug. 21, 2007. Priority is claimed on the following applications: Country: India, Application No. 1877/DEL/2006, Filed: Aug. 21, 2006; the content of which is incorporated here by reference.

### FIELD OF THE INVENTION

This invention relates to modified release pharmaceutical compositions and in particular to a modified release pharmaceutical composition of bupropion hydrochloride and a process for manufacturing same.

### BACKGROUND OF THE INVENTION

The hydrochloride salt of 1-(3-chlorophenyl)-2-[(1,1,-dimethylethyl)amino]-1-propanone, commonly known as bupropion hydrochloride, is disclosed in U.S. Pat. No. 3,819,706, assigned to Burroughs Wellcome. Antidepressant properties of bupropion hydrochloride are disclosed in U.S. Pat. No. 3,885,046. Bupropion hydrochloride is commercially available as immediate release tablets (75 and 100 mg) under the brand name Wellbutrin®, approved by the United States Food and Drug Administration (“FDA”) for major depressive disorders. Immediate release tablets suffer from the disadvantage that they induce severe side effects due to high peak plasma concentrations of bupropion. Bupropion is extensively metabolised in mammals and upon oral administration, only a small portion of the administered dose reaches the systemic circulation intact. Therefore, the development of extended controlled release dosage forms has been considered as an appropriate means to overcome the aforementioned problem.

Wellbutrin SR® and Wellbutrin XL®, commercially available from GlaxoSmithKline, are two currently marketed bupropion hydrochloride controlled release dosage forms. Wellbutrin XL® formulations are supplied for oral administration as 150 and 300 mg extended release tablets suitable for once a day administration.

Sustained release tablet forms of bupropion have been described in the prior art. U.S. Pat. No. 4,687,660, assigned to Burroughs Wellcome, discloses a tablet formed of a core and a coating, where the core comprises bupropion hydrochloride together with the excipients and optionally an osmotic enhancing agent and where the coating comprises a water-insoluble, water-permeable film-forming polymer, a pore-forming agent, and optionally a water permeability-enhancing agent and optionally a plasticizer.

U.S. Pat. Nos. 5,358,970 and 5,427,798, both assigned to Burroughs Wellcome, describe a sustained release formulation of bupropion hydrochloride based on a matrix technology. The term matrix refers to a tablet where the drug is embedded in an excipient that makes a non-disintegrating core called a matrix. Drug diffusion occurs through this core. As bupropion hydrochloride is unstable, the product described in the above two patents requires a stabilizer to achieve sufficient stability. The stabilizer is an acidic compound, preferably a cysteine hydrochloride. Sustained release tablets as described in U.S. Pat. No. 5,427,798 also provide peak bupropion blood levels at approximately 2-3 hours, thereby requiring twice daily dosing.

The FDA publication entitled “Approved Drug Products with Therapeutic Equivalence”, commonly referred to as the “Orange Book”, lists U.S. Pat. Nos. 6,096,341 and 6,143,327 as purportedly encompassing Wellbutrin XL® tablets.

U.S. Pat. Nos. 6,143,327 and 6,096,341, both assigned to Pharma Pass, disclose a delayed release coated tablet, free of stabilizer and free of pore-forming agent comprising a core consisting of bupropion hydrochloride, a binder and a lubricant and a first coating consisting essentially of a water-insoluble water-permeable film-forming polymer, a plasticizer and water-soluble polymer and a second coating essentially of a methacrylic acid polymer and a plasticizer.

U.S. Pat. No. 6,893,660, assigned to Andrx, discloses a pharmaceutical composition in solid form comprising pharmaceutically active ingredients combined with excipients having a negative effect on stability of the active ingredients by applying a sealing coating around the excipients having a negative effect. The invention requires a cumbersome and relatively expensive process of coating of the excipients. Depending on the excipients to be coated the process could be technically complex or difficult.

U.S. Pat. No. 6,306,436, assigned to Teva Pharmaceuticals, discloses stabilized bupropion hydrochloride pharmaceutical compositions that are free of added acid and provide for sustained release of bupropion hydrochloride. Stabilization is achieved by using particulate bupropion hydrochloride, which is coated with a membrane coating or by using large particle-size bupropion crystals. Although said patent avoids the potential disadvantages of using an acid, the disclosed invention requires drug particle coating, which may be an expensive and time-consuming process.

There is thus still a need for a modified release composition of bupropion, which is free of stabilizer and plasticizer and still achieves the desired dissolution profile and stability criteria of the FDA for being bioequivalent to the reference listed drug (Wellbutrin XL®).

### SUMMARY OF THE INVENTION

It is an object of the present invention to provide a delayed extended release pharmaceutical composition comprising bupropion or its pharmaceutically acceptable salts that exhibits predetermined extended release of the drug.

It is another object of the present invention to provide a process for manufacturing a delayed extended release composition comprising bupropion or its pharmaceutically acceptable salts that exhibits desired extended release of the drug.

Yet, another object of the present invention is to provide a delayed extended release composition comprising bupropion or its pharmaceutically acceptable salts which provides a therapeutically effective amount of bupropion such that the dosage form is administrable to a subject on a once-a-day basis.

Yet, another object of the present invention is to provide a delayed extended release pharmaceutical composition of bupropion or its pharmaceutically acceptable salts in such a way that it will comply with the reference product (Wellbutrin®XL) in terms of in vitro parameters like dissolution, disintegration, etc. and in vivo parameters like bioequivalence.

In accordance with yet another preferred embodiment of the present invention, there is provided a delayed extended release composition of bupropion or its pharmaceutically acceptable salts, wherein said composition is a once daily dosage form in the form of tablets of uniform size measuring approximately 6 to 12 mm.

According to one aspect of the present invention, there is provided a delayed extended release composition comprising (a) a compressed core comprising an effective amount of bupropion or its pharmaceutically acceptable salts, a water-attractant polymer and other pharmaceutically acceptable excipients, (b) one or more extended release layers surrounding said compressed core and/or coated core, and (c) one or more delayed release layers surrounding said extended release layered core, wherein said compressed core is devoid of any stabilizer. The invention in particular contemplates the application of alternate and repeated extended release and delayed release layers on said compressed core although the application of a single layer each of extended release and delayed release polymers is also within the scope of the invention.

In accordance with another preferred embodiment of the present invention, there is provided a delayed extended release composition of bupropion or its pharmaceutically acceptable salts, wherein said compressed core comprises about 50-98% by weight of bupropion or its pharmaceutically acceptable salts based on dry core weight, a water-attractant polymer from about 1 to about 10% by dry core weight and a lubricant from about 0.1 to about 5% by dry core weight.

In accordance with a preferred embodiment, the compressed core is coated with one or more extended release coating layers employing a rate-controlling polymer. The extended release coating layer consists essentially of an effective combination of pH-independent hydrophobic polymer and hydrophilic polymer, wherein said coating is characterized by the fact that it is essentially free of plasticizer and pore-forming agent.

In accordance with the invention, the compressed core is further coated with a delayed release layer employing an enteric polymer.

In accordance with a further preferred embodiment, the compressed core may have alternate multiple coatings of extended release and delayed release layer.

In accordance with still another preferred embodiment of the present invention, there is provided a delayed extended release pharmaceutical composition of bupropion or its pharmaceutically acceptable salts, wherein said composition is prepared by the process comprising the steps of: (1) dry mixing bupropion or its pharmaceutically acceptable salts and a water-attractant polymer, (2) granulating the resultant blend with aqueous/non aqueous solvent and drying the resultant granulates, (3) lubricating the dried granulates and compressing them into core tablets, (4) coating the core tablets with an extended release layer, (5) followed by coating the extended release layered tablets with a delayed release layer, and (6) repeating steps (4) and (5) to provide alternate extended release and delayed release layers on the tableted coated core.

Accordingly, it is an object of the present invention to produce a stable pharmaceutical composition of bupropion or its pharmaceutically acceptable salts suitable for once daily administration in the form of a compressed tablet dosage form, wherein said compressed core is devoid of any organic or inorganic stabilizing excipient and the extended release layer is essentially free of plasticizer and pore-forming agent.

It is also an object of the present invention to provide products that can be stored for long periods of time at room temperature, i.e., under humidity and temperature conditions usually encountered in pharmacies and medicine cabinets.

The pharmaceutical composition disclosed in accordance with the present invention is effectively used for the treatment of depression.

### DETAILED DESCRIPTION OF THE PRESENTLY PREFERRED EMBODIMENTS

The present invention provides a delayed extended release pharmaceutical composition of bupropion or its pharmaceutically acceptable salts and a process for manufacturing thereof.

Surprisingly, it has been found that in the bupropion hydrochloride composition of the present invention, use of a stabilizer in the core is essentially avoided, and also the addition of a plasticizer and pore-forming agents in the extended release layer is essentially avoided. Still the resulting products exhibit the desired release profile with optimum flexibility of the films along with excellent storage stability.

In one embodiment the composition of the present invention contains a compressed core which is free of a stabilizer and an extended release layer which is free of a pore-forming agent as well as a plasticizer and still, said compositions exhibit a dissolution profile and bioavailability substantially the same as the commercially available composition (Wellbutrin XL®) from GlaxoSmithKline.

The term ‘pharmaceutically acceptable salt of bupropion’ includes salts that are tolerated by a patient. Such salts are typically prepared from inorganic acids or bases and/or organic acids or bases. Examples of such acid and bases are well known to those of ordinary skill in the art. The invention in particular contemplates the use of bupropion hydrochloride, although the use of other pharmaceutically acceptable salts is within the scope of the invention.

The term ‘effective amount’ as used herein means a ‘pharmaceutically effective amount.’ A ‘pharmaceutically effective amount’ is the amount or quantity of the pharmaceutical acceptable salt of bupropion which is sufficient to elicit an appreciable biological response when administered to a patient.

The term ‘pharmaceutically acceptable excipient’ is intended to denote any material which is inert in the sense that it substantially does not have any therapeutic and/or prophylactic effect per se. Such an excipient may be added with the purpose of making it possible to obtain a pharmaceutical composition which has acceptable technical properties.

The term ‘stabilizer’ as used herein refers to compounds or a group of compounds capable of inhibiting or preventing degradation of bupropion hydrochloride in the dosage form.

The term ‘water-attractant polymer’ as used herein refers to any material which has the affinity for water thereby swelling, absorbing or being soluble in water.

The term ‘delayed extended release pharmaceutical composition’ as used herein refers to a solid oral dosage form which releases a drug at a particular time other than promptly after administration and allows a two-fold reduction in dosing frequency or increase in patient compliance or therapeutic performance.

The term ‘modified release pharmaceutical composition’ as used herein means a drug composition whose drug release characteristics with respect to time and/or location are chosen so as to accomplish therapeutic or convenience objectives not offered by conventional dosage forms.

According to the present invention, the term ‘delayed extended release pharmaceutical composition’ is used synonymously with the term ‘modified release pharmaceutical composition.’

‘Coating’ as used herein relates to the presence of a uniform film of polymeric material surrounding the drug-containing core. According to the present invention, the term ‘coating’ is used synonymously with the term ‘layer.’

‘Enteric coating’ as used herein relates to the presence of polymeric materials in a drug formulation that results in an increase in the drug's resistance to release in the stomach. According to the present invention, the term ‘enteric coating’ is used synonymously with the term “delayed release layer.”

It must be noted that, as used in the specification and the appended claims, the singular forms ‘a’, ‘an’ and ‘the’ include plural referents unless the context clearly dictates otherwise. Thus, for example, reference to ‘a coating’ includes multiple coatings.

‘Optional’ or ‘optionally’ means that the subsequently described circumstance may or may not occur, so that the description includes instances where the circumstance occurs and instances where it does not.

The term ‘about’ is used synonymously with the term ‘approximately’. Illustratively, the use of a term ‘about’ indicates that values slightly outside the cited values i.e. plus or minus 0.1 to 10%, which are also effective and safe.

In one aspect there is provided a delayed extended release pharmaceutical composition of bupropion or its pharmaceutically acceptable salts. The dosage form includes a core, and a coating on the core. The compressed core includes bupropion or its pharmaceutically acceptable salts, a water-attractant polymer and optionally one or more pharmaceutically acceptable excipients. The coating surrounding the core includes one or more extended release coating immediately surrounding the said core and/or coated core, followed by one or more delayed release coating immediately surrounding the extended release coating layered core.

In one general aspect, the present invention provides a process for preparing said delayed extended release pharmaceutical composition of bupropion hydrochloride. The steps of preparation include (1) preparing the core, (2) forming the extended release coating layer on the core, (3) forming the delayed release coating layer on the extended release coating layered core and (4) optionally repeating alternatively step (2) and (3).

In another general aspect, there is provided a process for the preparation of delayed extended release pharmaceutical composition of bupropion or its pharmaceutically acceptable salts. The process includes preparing a core, and coating the core with layers of extended release and delayed release polymers. The steps of preparation include (1) dry mixing bupropion or its pharmaceutically acceptable salt and water-attractant polymer, (2) granulating the resultant blend with aqueous/non-aqueous solvent and drying the resultant granulates, (3) lubricating the dried granules and compressing them into tablets, (4) coating the core tablets with a first extended release layer, (5) followed by coating the extended release layered tablets with a first delayed release layer, wherein said extended release and delayed release layers are repeatedly and alternatively applied on said compressed core. Furthermore, all the alternate extended release layers qualitatively have the same composition as the first extended release layer and all the alternate delayed release layers qualitatively have the same composition as the first delayed release layer.

### The Tablet Core

The term ‘core’ as used herein refers to any structure that is enclosed or surrounded by a coating. The core is preferably in the form of a compressed tablet.

The compressed core of the pharmaceutical composition of the present invention comprises bupropion or its pharmaceutically acceptable salts, one or more pharmaceutically acceptable excipients, including water-attractant polymers, and optionally other pharmaceutically acceptable excipients, wherein said core is devoid of stabilizer.

The bupropion or its pharmaceutically acceptable salt may be used in an amount from about 50 to about 98% by core dry weight, preferably from about 70 to about 98% by core dry weight. The preferred salt is the hydrochloride salt of bupropion.

The water-attractant polymers may include, but are not limited to one or more of polyvinylpyrrolidone, polyvinyl alcohol, hydroxypropyl cellulose, hydroxypropyl methylcellulose, modified starch, gelatin, sodium carboxymethyl cellulose, alginic acid or any combinations thereof. The preferred water-attractant polymer for the composition of the present invention is hydroxypropyl cellulose. Hydroxypropyl cellulose is commercially available under the brand name Klucel EXF Pharm from Aqualon Hercules, USA. Preferably, water-attractant polymer is used in an amount of about 1 to about 10% by core dry weight. The water-attractant polymer may also act as a binder and may also enhance the mechanical strength of the granules.

The compressed core of the present invention may further contain a lubricant. The non-limiting examples of lubricants used in the core of the pharmaceutical composition of the present invention include, but are not limited to magnesium stearate, stearic acid, glyceryl behenate, colloidon silicon dioxide, talc, calcium stearate, zinc stearate, polyethylene glycol, sodium stearyl fumarate, hydrogenated vegetable oil such as hydrogenated cottonseed oil, hydrogenated soyabean oil, glyceryl monostearate, glyceryl palmitostearate or any combination thereof. The preferred lubricant for the composition of the present invention is magnesium stearate. Magnesium stearate is commercially available from Mallinkrodt. Preferably, the lubricant is used in an amount of about 0.1 to about 5% by core dry weight.

The compressed core of the present invention may further contain fillers. Examples of fillers or diluents for use in the present invention include, but are not limited to lactose, microcrystalline cellulose, dextrin, dextrose, starch, mannitol and the like. The skilled artisan is aware of the regulatory requirements for hardness, friability, and disintegration time and can adjust the fillers using known techniques to achieve the desired physical characteristics.

The core of the present invention is highly water soluble in nature and behaves like an immediate release formulation at the uncoated stage. The additional inert excipients may be added to facilitate the preparation of the compressed core for the final delayed extended release composition of the present invention. The additional inert excipients are well known to the skilled artisan, for example anti-tacking agent and glidants such as colloidal silicon dioxide, talc, calcium silicate, and magnesium silicate.

The compressed core for the pharmaceutical composition of the present invention can be manufactured either by wet granulation, dry granulation or direct compression. Among the above-mentioned processes, wet granulation is the preferred one, in which the drug is mixed with water-attractant polymer and/or diluents, the premixed blend so obtained is granulated with aqueous/non-aqueous solvent in a rapid mixing granulator or fluid bed processor and dried in suitable dryer, for example retsch dryer or rapid dryer. The dried granular mass is milled and then mixed with other excipient like lubricants and if necessary any other additional inert excipients which may improve processing of the delayed extended release tablet of the present invention. The lubricated mass is then compressed into tablets using a tablet press (Cadmach, India).

The wet granulation may be done using aqueous/non-aqueous solvent. The aqueous solvent comprises purified water or the like, and the non-aqueous solvent comprises an organic solvent like ethanol, methanol, isopropyl alcohol, dichloromethane, glycols, acetone, ethyl acetate, dimethyl formamide, methylene chloride, benzene ethyl lactate, glacial acetic acid, etc.

### Extended Release Layer

An extended release layer for the composition of the present invention refers to one or more layers having a combination of a pH independent hydrophobic polymer and hydrophilic polymer.

The extended release layer consists essentially of pH independent hydrophobic polymer and hydrophilic polymer wherein said layer is essentially free of any pore-forming agents and plasticizers.

The term ‘consisting essentially of’, in one embodiment, is intended to mean that the listed compounds represent more than 90% by weight of the extended release layer, preferably more than 95% by weight of the extended release layer, most preferably more than 98% by weight of the extended release layer.

As used herein, the phrase ‘essentially free of any pore-forming agent and plasticizer’ means that a plasticizer and a pore-forming agent are not used during the preparation of the extended release layer of the composition of the present invention.

The first extended release layer is applied directly onto the surface of the compressed core and functions to modulate the release of the pharmaceutically acceptable salt of bupropion. Thereafter, one or more extended release layer may be alternatively or repeatedly applied over said enteric-coated tablet. In the preferred embodiment, the core is covered with two or more extended and delayed release layers.

Non-limiting examples of pH-independent hydrophobic polymers for the extended release layer include cellulose ether, such as ethylcellulose, cellulose ester, such as cellulose acetate, polyvinylalcohol, copolymers of acrylate and methacrylates with quarternary ammonium groups such as, Eudragit®RL and Eudragit®RS (available from Röhm Pharma). The preferred pH-independent hydrophobic polymer is ethylcellulose (available from Dow Chemical and Hercules under the trade name Ethocel® and Aqualon® respectively). With respect to the extended release layer itself, the pH-independent hydrophobic polymer is preferably used in an amount of from about 20 to about 50% by weight of the extended release layer dry weight.

Suitable hydrophilic polymers for the extended release layer include, but are not limited to polyvinylpyrrolidone, poly(ethylene oxide), hydroxypropyl methylcellulose and hydroxypropyl cellulose. The preferred hydrophilic polymer is hydroxypropyl cellulose. Hydroxypropyl cellulose is commercially available under the brand name Klucel EXF Pharm from Aqualon Hercules, USA. With respect to the extended release layer itself, hydrophilic polymer is preferably used in an amount of from about 40 to about 80% by weight of the extended release layer dry weight.

Depending on the dissolution profile desired, the weight gained with the extended release layer(s) might vary from about 3 to 20% of the weight of the dry tablet core. Preferably, the weight gain after the extended release layer is approximately 5 to 10% of the weight of the dry tablet core.

The ratio of pH-independent hydrophobic polymer and hydrophilic polymer present in the extended release layer ranges from 1:3 to 3:1. The skilled artisan will appreciate that varying the ratio of hydrophilic and hydrophobic polymer can modulate the release of bupropion from the core. A more extended release is generally obtained with a higher amount of pH-independent hydrophobic polymer.

An exemplary composition for the extended release layer comprises by weight, based on the dried weight of said layer: about 20% to about 50%, preferably about 25% to about 40%, of ethylcellulose; about 40% to about 80%, preferably about 50% to about 75%, of hydroxypropyl cellulose (or another polymer such as hydroxypropyl methylcellulose).

The dispersion/solution of pH-independent hydrophobic polymer and hydrophilic polymer may be prepared in an aqueous/non-aqueous solvent. The aqueous solvent comprises purified water or the like, and non-aqueous solvent comprises organic solvent like ethanol, methanol, isopropyl alcohol, dichloromethane, glycols, acetone, ethyl acetate, dimethyl formamide, methylene chloride, benzene ethyl lactate, glacial acetic acid, etc. The extended release layer may optionally contain buffers, colorants, opacifiers, surfactants, which are known to those skilled in the art. The extended release layer can be applied onto the tablet cores using a conventional coating pan, perforated coating pan, fluidized bed apparatus or any other suitable coating apparatus known to achieve the desired weight gain. Processing parameters of the above-mentioned coating equipments are well known to the person skilled in the pharmaceutical art.

### Delayed Release Layer

The delayed release layer for the composition of the present invention refers to one or more layers comprising an enteric polymer and produced on to the extended release layer.

The delayed release layer for the pharmaceutical composition of the present invention consists essentially of a methacrylic acid copolymer or hydroxypropyl methylcellulose phthalate, a plasticizer, a pore-forming agent and an anti-tacking agent.

The term ‘consists or consisting essentially of’, in one embodiment is intended to mean that the listed compounds represent more than 90% by weight of the delayed release layer, preferably more than 95% by weight of the delayed release layer, most preferably more than 98% by weight of the delayed release layer.

Supplementary agents such as opacifiers, coloring agents as well as any other excipient that conventionally enters into the composition of enteric coatings can be employed in the delayed release layer of the present invention.

The enteric polymers of the present invention includes methacrylic acid copolymer and hydroxypropyl methylcellulose phthalate. Anionic polymers with methacrylic acid as a functional group are excellent enteric polymers and are commercially available from Degussa, Germany, under the brand names Eudragit®L 100-55 (powder form), Eudragit®L30D-55 (30% aqueous dispersion of Eudragit®L 100-55), Eudragit®L 100 (powder form) and Eudragit®S 100 (powder form). Still more preferred enteric polymers for the composition of the present invention are Eudragit®L30D-55 which is an aqueous dispersion of anionic copolymer based on methacrylic acid and ethylacrylate, and hydroxypropyl methylcellulose phthalate. Hydroxypropyl methylcellulose phthalate is commercially available from ShinEtsu, Japan, under the brand name HP-50 and HP-55. Those skilled in the art will appreciate that the amount of enteric polymer may vary depending on the strength of particular polymer used and the level approved by regulatory authorities for use in pharmaceutical products. Suitable methacrylic acid copolymer and hydroxypropyl methylcellulose phthalate are used in an amount from about 30% to about 90% by weight of the delayed release layer dry weight.

The non-limiting examples of plasticizers include triethyl citrate, tributyl citrate, triacetin, polyethylene glycol, propylene glycol, diethylphthatate and oils/glycerides such as fractionated coconut oil or castor oil and any combination thereof. The preferred plasticizer is a combination of triethyl citrate and polyethylene glycol. Triethyl citrate is commercially available under the brand name Citroflex from Pfizer, USA. Triethyl citrate is also commercially available from Morflexi Inc, USA. Polyethylene glycol is commercially available under the brand name Macrogol from Sanyo Ind, Japan. Polyethylene glycol is commercially available under the brand name Lutrol from BASF Aktiengesellschaft. The expert knows, on the basis of his technical knowledge, various percentages and ranges of plasticizers that can be used in conjunction with enteric polymers. A suitable plasticizer is used in an amount of from about 5% to about 30% by weight of the delayed release layer dry weight.

Examples of pore-forming agents for the delayed release layer include lactose, sucrose, sorbitol, mannitol, sodium chloride, calcium chloride, potassium chloride and the like. The range of channeling agents in the formulations depends on the type of composition prepared and will be apparent to one skilled in the art based on the various examples disclosed in the specification. A suitable pore-forming agent is used in an amount of from about 5 to about 30% by weight of the delayed release layer dry weight.

Non-limiting examples of anti-tacking agents include calcium stearate, colloidal silicon dioxide and talc. The preferred anti tacking agent is talc. Talc is commercially available from Luzenac, Italy. A suitable anti-tacking agent is used in an amount of from about 5 to about 30% by weight of the delayed release layer dry weight.

The artisan can choose an appropriate opacifier known in the art, for example, titanium dioxide. The preferred opacifier for the delayed release layer is titanium dioxide.

The first delayed release layer is applied directly onto the extended release coating layered tablet. Thereafter, one or more delayed release layer may be alternatively or repeatedly applied over said extended release coating layered tablet. Furthermore, the compositions of the delayed release layers are qualitatively similar to each other, for instance first delayed release layer is qualitatively similar to second delayed release layer. In the preferred embodiment, the core is covered with two alternate delayed release layers.

An exemplary composition for the delayed release layer comprises by weight, based on the weight of said layer: about 30% to about 90%, preferably about 35% to about 85%, of methacrylic acid copolymer (or another enteric polymer); about 3% to about 25%, preferably about 5% to about 15%, of triethyl citrate (or another plastfying agent); about 3% to about 25%, preferably about 5% to about 15%, of polyethylene glycol; about 5% to about 30%, preferably about 10% to about 20% of lactose; about 5% to about 30%, preferably about 10% to about 20%, of talc.

Depending on the dissolution profile desired the weight gained after coating tablet core with the delayed releasing coat(s) might vary from about 5 to about 25% of the weight of the dry tablet core. Preferably, the weight gain is approximately 5 to 15% of the weight of the dry tablet core.

The dispersion of enteric polymer may be prepared in an aqueous/non-aqueous solvent. The aqueous solvent comprises purified water or the like, and non-aqueous solvent comprises organic solvent like ethanol, methanol, isopropyl alcohol, dichloromethane, glycols, acetone, ethyl acetate, dimethyl formamide, methylene chloride, benzene ethyl lactate, glacial acetic acid etc. The delayed release layer may further optionally contain buffers, colorants, surfactants, which are known to those skilled in the art. The delayed release layer can be applied onto the extended release layer using a conventional coating pan, perforated coating pan, fluidized bed apparatus or any other suitable coating apparatus known in the art to achieve the desired weight gain. The skilled artisan knows, on the basis of his technical knowledge the optimization techniques of various processing parameters for the above-mentioned coating equipments.

The present invention also provides a process for the preparation of a pharmaceutical composition of bupropion or its pharmaceutically acceptable salts thereof as active ingredient, comprising the steps of: (1) dry mixing bupropion or its pharmaceutically acceptable salt and water attractant polymer; (2) granulating the resultant blend with aqueous/non aqueous solvent; (3) milling the wet mass as obtained in step (2) through a 0.475 mm screen and drying the so formed granulates; (4) passing the resultant granules through a 0.710 mm screen and mixing the same with lubricants and glidants; (5) compressing the lubricated granules into tablet core; (6) preparing an extended release coating solution by dispersing or dissolving a pH independent hydrophobic polymer and hydrophilic polymer in the aqueous/non-aqueous solvent system; (7) layering an extended release layer on the core as obtained in step (5) with the coating solution as obtained in step (6); (8) preparing a delayed release coating dispersion/solution by dispersing methaerylic acid copolymer in aqueous/non-aqueous solvent system and adding plasticizer, anti tacking agent, pore-forming agent and opacifier into said dispersion; (9) layering the delayed release coat on the extended release coated core as obtained in step (7) with the delayed release coating dispersion/solution as obtained in step (8); and (10) repeating steps (6) to (10) to apply alternate extended release and delayed release layers on the tableted coated core.

The preferred weight of the tablets of the present invention is 100 to 1000 mg, more preferably 150 to 500 mg.

For administration of bupropion hydrochloride to treat depression, a typical daily dose is in the range of approximately 50 to 500 mg, preferably 100 to 450 mg. When used as an aid in smoking cessation, the typical daily dose is in the range of approximately 150 to 300 mg. However, the exact dosage regimen will depend on a number of factors, including age, the general condition of a patient, the particular condition or disorder being treated, the severity of the patient's condition or disorder, and the like.

The positive impact on stability of the delayed extended release bupropion hydrochloride tablet described herein is evident from the results of tests performed to evaluate the total related substances present in 150 mg pharmaceutical composition through 3 months under accelerated conditions (40° C.±2° C./75% RH±5% RH). The stability tests showed reduced values in total related substances in tablets. The delayed extended release tablets of bupropion hydrochloride of the present invention disclosed herein contain at least about 90% of bupropion hydrochloride after three months storage at about 40° C.±2° C./75% RH±5% RH.

The following examples will further illustrate certain specific aspects and embodiments of the invention in greater detail and are not to be construed as limiting the scope of the invention.

### Example 1

### Delayed Extended Release Pharmaceutical Composition of Bupropion Hydrochloride

TABLE 1

| | | |
|---|---|---|
| S. No | Ingredients | Quantity (mg/tab) |

a) Core composition

| | | |
|---|---|---|
| 1. | Bupropion hydrochloride | 150 |
| 2. | Hydroxypropyl cellulose | 4.6 |
| 3. | Magnesium stearate | 2.4 |
| 4. | Isopropyl alcohol* | q.s |

b) Extended release coating layer

| | | |
|---|---|---|
| 5. | Ethyl cellulose | 2.5 |
| 6. | Hydroxypropyl methylcellulose 6 cps | 1.9 |
| 7. | Hydroxypropyl methylcellulose 3 cps | 1.9 |
| 8. | Isopropyl alcohol* | q.s |
| 9. | Dichloromethane* | q.s |

c) Delayed release coating layer

| | | |
|---|---|---|
| 10. | Eudragit L30D 55 (solid content) | 5.1 |
| 11. | Triethyl citrate | 0.5 |
| 12. | Polyethylene glycol 400 | 1.0 |
| 13. | Lactose | 1.3 |
| 14. | Talc | 1.0 |
| 15. | Purified water* | q.s |

*Not present in the final composition

### Manufacturing Procedure:

### (a) Preparation of the Core

(I) Bupropion hydrochloride was passed through a 1 mm screen.
(II) Hydroxypropyl cellulose was dissolved in isopropyl alcohol.
(III) Bupropion hydrochloride was granulated with the solution obtained in step (II) above in a rapid mixing granulator.
(IV) The granulate obtained in step (III) was dried in a Retsch dryer at 55° C. for 7 to 10 minutes.
(V) The dried granules as obtained in step (IV) were passed through a 0.6 mm screen and mixed with magnesium stearate, which was previously passed through a 0.25 mm screen.
(VI) The lubricated granules as obtained in step (V) were compressed to give the core in the form of a tablet.

### (b) Preparation and Application of Extended Release Coating Layer on the Core

(VII) Ethylcellulose, hydroxypropyl methylcellulose 6 cps and hydroxypropyl methylcellulose 3 cps were dissolved in the mixture of isopropyl alcohol and dichloromethane.
(VIII) The coating solution as obtained above in step (VII) was coated on the core of step (VI) in a conventional coating pan under appropriate coating conditions such as inlet air temperature: 50-55° C.; exhaust air temperature 35-36° C.; pan speed: RPM: 6-8; pump speed: RPM 5-7.

### (c) Preparation and Application of Delayed Release Coating Layer on the Extended Release Coating Layered Core

(IX) Triethyl citrate and polyethylene glycol was dissolved in 60% of the water and said solution was added to the Eudragit polymer.
(X) Talc was added to the remaining 40% of water, and the dispersion so formed was homogenized.
(XI) The solutions obtained in steps (IX) and (X) were blended together,
(XII) The solution obtained in step (XI) was coated on the extended release coated core of step (VIII) in a conventional coating pan under appropriate coating parameters such as inlet air temperature: 70-75° C.; exhaust air temperature 40-42° C.; pan speed: RPM: 6-8; pump speed: RPM: 2-4.

### Example 2

### Delayed Extended Release Pharmaceutical Composition of Bupropion Hydrochloride

TABLE 2

| | | |
|---|---|---|
| S. No | Ingredients | Quantity (mg/tab) |

a) Core composition

| | | |
|---|---|---|
| 1. | Bupropion hydrochloride | 150 |
| 2. | Hydroxypropyl cellulose | 4.6 |
| 3. | Magnesium stearate | 2.4 |
| 4. | Isopropyl alcohol* | q.s |

b) Extended release coating layer I

| | | |
|---|---|---|
| 5. | Ethyl cellulose | 2.6 |
| 6. | Hydroxypropyl cellulose | 5.3 |
| 7. | Isopropyl alcohol* | q.s |
| 8. | Dichloromethane* | q.s |

c) Delayed release coating layer I

| | | |
|---|---|---|
| 9. | Eudragit L30D 55 (solid content) | 6.63 |
| 10. | Triethyl citrate | 0.7 |
| 11. | Polyethylene glycol 400 | 1.3 |
| 12. | Lactose | 1.7 |
| 13. | Talc | 1.3 |
| 14. | Purified water* | q.s |

d) Extended release coating layer II

| | | |
|---|---|---|
| 15. | Ethyl cellulose | 1.1 |
| 16. | Hydroxypropyl cellulose | 1.6 |
| 17. | Isopropyl alcohol* | q.s |
| 18. | Dichloromethane* | q.s |

e) Delayed release coating layer II

| | | |
|---|---|---|
| 19. | Eudragit L30D 55 (solid content) | 2.1 |
| 20. | Triethyl citrate | 0.21 |
| 21. | Polyethylene glycol 400 | 0.42 |
| 22. | Lactose | 0.51 |
| 23. | Talc | 0.41 |
| 24. | Purified water* | q.s. |

*Not present in the final composition

### Manufacturing Procedure:

### (a) Preparation of the Core

(I) Bupropion hydrochloride and hydroxypropyl cellulose were passed through a 1 mm screen and then blended together.
(II) The blend of stage (I) was granulated with isopropyl alcohol in a rapid mixing granulator.
(III) The granulate obtained in step (II) was milled through a 0.475 mm screen and dried in a rapid dryer at 50° C. for 5 to 7 minutes.
(IV) The dried granules as obtained in step (III) were passed through a 0.710 mm screen and mixed with magnesium stearate, which was previously passed through a 0.25 mm screen.
(V) The lubricated granules as obtained in step (IV) were compressed to give the core tablet.

### (b) Preparation and Application of Extended Release Coating Layer I on the Core

(VI) Ethylcellulose, and hydroxypropyl cellulose were dissolved in a mixture of isopropyl alcohol and dichloromethane.
(VII) The coating solution as obtained above in step (VI) was coated on the core of step (V) in a perforated coating pan under appropriate coating parameters such as: inlet air temperature: 41-42° C.; exhaust air temperature: 35-36° C.; pan speed: RPM: 4-6; peristaltic pump speed: RPM: 12-16.

### (c) Preparation and Application of Delayed Release Coating Layer I on the Extended Release Coating Layered Core

(VIII) Triethyl citrate, polyethylene glycol 400 and lactose were dissolved in half of the purified water.
(IX) Eudragit L 30D55 was added to the solution as obtained in step (VIII).
(X) Talc was added to the remaining 40% of water, and the dispersion so formed was homogenized.
(XI) The solutions obtained in steps (IX) and (X) were blended together.
(XII) The solution obtained in step (XI) was coated on the extended release coated core of step (VII) in a perforated coating pan and under appropriate coating parameters including: inlet air temperature: 47-49° C.; exhaust air temperature: 40-41° C.; pan speed: RPM: 5-7; peristaltic pump speed: RPM: 10-12.

### (d) Preparation and Application of Extended Release Coating Layer II on the Delayed Release Layered Core

(XIII) Same procedure was followed as mentioned in steps (VI) and (VII).

### (e) Preparation and Application of Delayed Release Coating Layer II on the Extended Release Coating Layered Core

(XIV) Same procedure was followed as mentioned in steps (VIII) to (XII).

### Example 3

### Delayed Extended Release Pharmaceutical Composition of Bupropion Hydrochloride

TABLE 3

| | | |
|---|---|---|
| S. No | Ingredients | Quantity (mg/tab) |

a) Core composition

| | | |
|---|---|---|
| 1. | Bupropion hydrochloride | 150 |
| 2. | Hydroxypropyl cellulose | 4.6 |
| 3. | Magnesium stearate | 2.4 |
| 4. | Isopropyl alcohol* | q.s |

b) Extended release coating layer I

| | | |
|---|---|---|
| 5. | Ethyl cellulose | 2.33 |
| 6. | Hydroxypropyl cellulose | 4.74 |
| 7. | Isopropyl alcohol* | q.s |
| 8. | Dichloromethane* | q.s |

c) Delayed release coating layer I

| | | |
|---|---|---|
| 9. | Eudragit L30D 55 (solid content) | 7.0 |
| 10. | Triethyl citrate | 0.7 |
| 11. | Polyethylene glycol 400 | 1.4 |
| 12. | Lactose | 1.75 |
| 13. | Talc | 1.4 |
| 14. | Purified water* | q.s |

d) Extended release coating layer II

| | | |
|---|---|---|
| 15. | Ethyl cellulose | 1.1 |
| 16. | Hydroxypropyl cellulose | 1.6 |
| 17. | Isopropyl alcohol* | q.s |
| 18. | Dichloromethane* | q.s |

e) Delayed release coating layer II

| | | |
|---|---|---|
| 19. | Eudragit L30D 55 (solid content) | 2.0 |
| 20. | Triethyl citrate | 0.2 |
| 21. | Polyethylene glycol 400 | 0.4 |
| 22. | Lactose | 0.51 |
| 23. | Talc | 0.4 |
| 24. | Purified water* | q.s |

*Not present in the final composition

### Manufacturing Procedure

### (a) Preparation of the Core

(I) Bupropion hydrochloride was passed through a 1 mm screen.
(II) Hydroxypropyl cellulose was dissolved in isopropyl alcohol.
(III) Bupropion hydrochloride was granulated with the solution obtained in step (II) above in a rapid mixing granulator.
(IV) The granulate obtained in step (III) was dried in a Retsch dryer at 50° C. for 5 to 7 minutes.
(V) The dried granules as obtained in step (IV) passed through a 0.710 mm screen and mixed with magnesium stearate, which was previously passed through a 0.25 mm screen.
(VI) The lubricated granules as obtained in step (V) were compressed to give a core in the form of a tablet.

### (b) Preparation and Application of Extended Release Coating Layer I on the Core

(VII) Ethylcellulose, and hydroxypropyl cellulose were dissolved in a mixture of isopropyl alcohol and dichloromethane.
(VIII) The coating solution as obtained above in step (VII) was coated on the core of step (VI) in a perforated coating pan under appropriate coating parameters such as: inlet air temperature: 45-50° C.; exhaust air temperature: 35-36° C.; pan speed: RPM: 4-6; peristaltic pump speed: RPM: 2-3.

### (c) Preparation and Application of Delayed Release Coating Layer I on the Extended Release Coating Layered Core

(IX) Triethyl citrate, polyethylene glycol 400 and lactose were dissolved in a suitable quantity of purified water.
(X) Eudragit L 30D55 was added to the solution as obtained in step (IX).
(XI) Talc was added to the remaining purified water, and the dispersion so formed was homogenized.
(XII) The solutions obtained in steps (X) and (XI) were blended together.
(XIII) The solution obtained in step (XII) was coated on the extended release coated core of step (VIII) in a perforated coating pan and under appropriate coating parameters such as inlet air temperature: 65-70° C.; exhaust air temperature: 39-40° C.; pan speed: RPM: 5-7; peristaltic pump speed: RPM: 1-2.

### (d) Preparation and Application of Extended Release Coating Layer II on the Delayed Release Layered Core

(XIV) Same procedure was followed as mentioned in steps (VII) and (VIII).

### (e) Preparation and Application of Delayed Release Coating Layer II on the Extended Release Coating Layered Core

(XV) Same procedure was followed as mentioned in steps (IX) to (XIII).

### Example 4

To assess the release of drug substance (bupropion hydrochloride) from the drug product or dosage form, coated tablets of Example 2 were subjected to a dissolution study. The dissolution profile from coated tablets of Example 2 was compared with the dissolution profile from commercially available bupropion extended release tablets (Wellbutrin XL® 150 mg) from Glaxosmithkilne, USA. The results are presented in table 4 as the mean percentage release of the total bupropion hydrochloride contents from the coated tablets. Dissolution study parameters were as follows:

Instrument parameters: USP type I; 75 RPM
Dissolution medium parameters: 0.1 N HCl; 900 ml; 37° C.±2° C.

TABLE 4

Mean percentage release of drug (bupropion)

| Time (hr) | Wellbutrin XL ® | Coated tablets of Example 2 |
|---|---|---|
| 1 | 0 | 2 |
| 2 | 1 | 2 |
| 4 | 22 | 40 |
| 6 | 50 | 75 |
| 8 | 78 | 88 |
| 10 | 89 | 95 |
| 12 | 94 | 100 |
| 16 | 99 | 101 |
| 20 | 101 | 101 |
| 24 | 102 | 103 |

From the above tabular data it is clearly evident that the coated tablets of the invention (Ex. 2) have substantially the same dissolution profile as the Wellbutrin XL® tablets.

### Example 5

In order to assess the stability of drug substance (bupropion hydrochloride) in the drug product or dosage form, coated tablets of Example 3 were subjected to accelerated stability testing at 40° C.±2° C./75% RH±5% RH and observations were made during and after 3 months in HDPE bottles for percentage of unreacted bupropion and total related substances (RS) analyzed by validated high performance liquid chromatography. The results are shown in Table 5 below.

TABLE 5

| | (%) Bupropion HCL | |
|---|---|---|
| Study period | present | Total RS |
| Initial | 99.2 | 0.16 |
| After 1 month | 98.1 | 0.10 |
| After 2 months | 100.3 | 0.09 |
| After 3 months | 99.0 | 0.17 |

From the above tabular data it is clearly evident that the coated tablets of the invention (Example 3) have a negligible amount of % w/w of total related substances in the drug product.

It is to be understood that while the invention has been described in conjunction with the preferred specific embodiments thereof, that the description and examples given above are intended to illustrate and not to limit the scope of the invention. Other aspects, advantages and modifications within the scope of the invention will be apparent to those skilled in the art to which the invention pertains. Accordingly, the appended claims are intended to cover all embodiments of the invention and modifications thereof that do not depart from the spirit and scope of the invention.

1.-34. (canceled)

35. A delayed extended release pharmaceutical composition comprising:
(a) a compressed core comprising an effective amount of bupropion or its pharmaceutically acceptable salt, and a water-attractant polymer;
(b) an extended release layer surrounding said core, wherein said layer is essentially free of plasticizer and pore-forming agent; and
(c) a delayed release layer surrounding said extended release layered core.

36. The composition of claim 35, wherein the compressed core further comprises one or more pharmaceutically acceptable excipients.

37. A delayed extended release pharmaceutical composition comprising:
(a) a compressed core comprising an effective amount of bupropion or its pharmaceutically acceptable salt, and a water-attractant polymer, wherein said core is devoid of a stabilizer;
(b) a first extended release layer surrounding said core, wherein said layer is essentially free of plasticizer and pore-forming agent;
(c) a first delayed release layer surrounding said extended release layered core;
(d) a second extended release layer surrounding said delayed release layered core; and
(e) a second delayed release layer surrounding said extended release layered core.

38. The composition of claim 37, wherein the compressed core further comprises one or more pharmaceutically acceptable excipients.

39. The composition of claim 37, wherein the composition further comprises one or more additional extended and delayed release layers.

40. A delayed extended release pharmaceutical composition comprising:
(a) a compressed core comprising an effective amount of bupropion or its pharmaceutically acceptable salts, hydroxypropyl cellulose and magnesium stearate, wherein said core is devoid of a stabilizer;
(b) a first extended release layer surrounding said core, said layer consisting essentially of ethylcellulose and hydroxypropyl cellulose or hydroxypropyl methylcellulose, wherein said layer is essentially free of plasticizer and pore-forming agent;
(c) a first delayed release layer surrounding said extended release layered core, said layer consisting essentially of methacrylic acid copolymer or hydroxypropyl methylcellulose phthalate, lactose, and a combination of triethyl citrate and polyethylene glycol and talc;
(d) a second extended release layer surrounding said delayed release layered core, wherein said extended release layer consists essentially of ethylcellulose and hydroxypropyl cellulose or hydroxypropyl methylcellulose, wherein said layer is essentially free of plasticizer and pore-forming agent; and
(e) a second delayed release layer surrounding said extended release layered core, wherein said delayed release layer consists essentially of methacrylic acid copolymer or hydroxypropyl methylcellulose phthalate, lactose, and a combination of triethyl citrate and polyethylene glycol and talc.

41. The pharmaceutical composition according to claim 35, wherein said pharmaceutically acceptable salt of bupropion is used in the amount of from about 50 to about 98% by core dry weight.

42. The pharmaceutical composition according to claim 35, wherein said water-attractant polymer is used in an amount of from about 1 to about 10% by core dry weight.

43. The pharmaceutical composition according to claim 35, wherein the said water-attractant polymer present in the core comprises polyvinylpyrrolidone, polyvinyl alcohol, hydroxypropyl cellulose, hydroxypropyl methylcellulose, modified starch, gelatin, sodium carboxymethyl cellulose, alginic acid or any combination thereof.

44. The pharmaceutical composition according to claim 43, wherein the preferred water-attractant polymer is hydroxypropyl cellulose.

45. The pharmaceutical composition according to claim 35, wherein the core of said composition further comprises a lubricant and a glidant.

46. The pharmaceutical composition of claim 45, wherein said lubricant comprises magnesium stearate, stearic acid, glyceryl behenate, colloidal silicon dioxide, talc, calcium stearate, zinc stearate, polyethylene glycol, sodium stearyl fumarate, light mineral oil, hydrogenated vegetable oil such as hydrogenated cottonseed oil, hydrogenated soyabean oil or any combination thereof.

47. The pharmaceutical composition of claim 46, wherein said lubricant is magnesium stearate.

48. The pharmaceutical composition according to claim 45, wherein the said glidant comprises calcium silicate, magnesium silicate, colloidal silicon dioxide or talc.

49. The pharmaceutical composition of claim 48, wherein said glidant is talc.

50. The pharmaceutical composition according to claim 35, wherein said extended release layer consists essentially of a pH-independent hydrophobic polymer and a hydrophilic polymer; and wherein said layer is essentially free of plasticizer and pore-forming agent.

51. The pharmaceutical composition according to claim 50, wherein said pH-independent hydrophobic polymer is present at about 20 to 50% by weight of the extended release layer dry weight.

52. The pharmaceutical composition according to claim 50, wherein said pH-independent hydrophobic polymer comprises ethylcellulose, cellulose acetate or polyvinylalcohol or copolymers of acrylate and methacrylates with a quarternary ammonium group, or any combination thereof.

53. The pharmaceutical composition according to claim 52, wherein said pH-independent hydrophobic polymer is ethylcellulose.

54. The pharmaceutical composition according to claim 50, wherein said hydrophilic polymer is present at about 40 to about 80% by weight of the extended release layer dry weight.

55. The pharmaceutical composition according to claim 50, wherein said hydrophilic polymer present in said extended release layer comprises hydroxypropyl cellulose, polyvinylpyrrolidone, polyethylene oxide, hydroxymethyl cellulose, hydroxypropyl methylcellulose or any combination thereof.

56. The pharmaceutical composition according to claim 55, wherein said hydrophilic polymer is hydroxypropyl cellulose.

57. The pharmaceutical composition according to claim 50, wherein the ratio of the pH independent hydrophobic polymer to hydrophilic polymer is about from 1:3 to 3:1.

58. The pharmaceutical composition according to claim 50, exhibiting a weight gain after application of the extended release layer from about 3 to 20% of the weight of the dry core.

59. The pharmaceutical composition according to claim 35, wherein said delayed release layer consists essentially of a methacrylic acid copolymer or hydroxypropyl methylcellulose phthalate, plasticizer, pore-forming agent and anti-tacking agent.

60. The pharmaceutical composition according to claim 59, wherein said methacrylic acid copolymer is present at about 30% to 90% by weight of the delayed release layer dry weight.

61. The pharmaceutical composition according to claim 59, wherein said plasticizer is present at about 5% to 30% by weight of the delayed release layer dry weight.

62. The pharmaceutical composition according to claim 59, wherein said plasticizer comprises triethyl citrate, tributyl citrate, triacetin, polyethylene glycol, propylene glycol, diethylphthatate and oils/glycerides comprising fractionated coconut oil or castor oil, or any combination thereof.

63. The pharmaceutical composition according to claim 62, wherein said plasticizer is a combination of triethyl citrate and polyethylene glycol.

64. The pharmaceutical composition according to claim 59, wherein said pore-forming agent is present at about 5% to 30% by weight of the delayed release layer dry weight.

65. The pharmaceutical composition according to claim 59, wherein said pore-forming agent comprises lactose, sucrose, sorbitol, mannitol, sodium chloride, calcium chloride, potassium chloride or any combination thereof.

66. The pharmaceutical composition according to claim 65, wherein said pore-forming agent is lactose.

67. The pharmaceutical composition according to claim 59, wherein said anti-tacking agent comprises calcium stearate, colloidal silicon dioxide and talc.

68. The pharmaceutical composition according to claim 67, wherein said anti-tacking agent is talc.

69. The pharmaceutical composition according to claim 59, wherein the composition exhibits a weight gain after application of said delayed release layer from about 5 to 25% of the weight of the dry tablet core.

70. The pharmaceutical composition as claimed in claim 35, wherein said composition is prepared by a process comprising the steps of:
(1) dry mixing bupropion or its pharmaceutically acceptable salt and water attractant polymer; (2) granulating the resultant blend with aqueous/non aqueous solvent; (3) milling the wet mass as obtained in step (2) through a 0.475 mm screen and drying the so formed granulates; (4) passing the resultant granules through a 0.710 mm screen and mixing the same with lubricants and glidants; (5) compressing the lubricated granules into a tablet core; (6) preparing an extended release coating solution by dispersing a pH-independent hydrophobic polymer and hydrophilic polymer in a isopropyl alcohol and dichloromethane based solvent system; (7) layering an extended release layer on the core as obtained in step (5) with the coating solution as obtained in step (6); (8) preparing a delayed release coating dispersion by dispersing methacrylic acid copolymer in purified water; (9) layering a delayed release coat on the extended release coated core as obtained in step (7) with the delayed coating dispersion obtained in step (8); and (10) repeating steps (6) to (10) to apply alternate extended release and delayed release layers on the tableted coated core.

71. The pharmaceutical composition according to claim 70, wherein a plasticizer, anti tacking agent, pore-forming agent and opacifier are added to said dispersion in step (8).
